(19) United States
(12) Patent Application Publication
(10) Pub. No.: US 2004/0092413 A1
(43) Pub. Date: May 13, 2004

(54) Concentrated liquid compositions and methods of providing the same

(75) Inventor: Richard Ticktin, Ft. Lauderdale, FL (US)

Correspondence Address: BOZICEVIC, FIELD & FRANCIS LLP, 200 MIDDLEFIELD RD, SUITE 200, MENLO PARK, CA, 94025, US

(73) Assignee: SynergyLabs

(21) Appl. No.: 10/631,025

(22) Filed: Jul. 29, 2003

Related U.S. Application Data
(60) Provisional application No. 60/399,552, filed on Jul. 29, 2002.

(51) Int. Cl. A61K 7/50

(52) U.S. Cl. 510/130

(57) ABSTRACT

A concentrated composition and methods for providing the same are disclosed. The concentrated liquid composition includes a suspension matrix and at least one surfactant dispersed in the suspension matrix, wherein the suspension matrix has a concentration of water less than about 5% by volume.

### CROSS-REFERENCE

This application claims the benefit of U.S. Provisional Application No. 60/399,552, filed Jul. 29, 2002, which application is incorporated herein by reference.

### TECHNICAL FIELD

This invention relates generally to concentrated compositions and methods of providing the same. In particular the concentrated detergent compositions and methods relate to super-concentrated compositions, including shampoos, having less than about 5% by weight of water prior to dilution which are dilutable to provide a ready-to-use cleanser or detergent.

### BACKGROUND OF THE INVENTION

Liquid detergents enjoy wide consumer acceptance for numerous uses. Liquid detergents are preferred over dry granular detergents because liquid detergents can be used without having to pre-dissolve the detergent. Moreover, liquid detergents can be directly applied to stains and fabrics. The water content of these liquid detergents is approximately 90% to 95%. Many of these detergents are packaged in gallon-size containers, particularly for commercial use, and as such, are heavy (weighing approximately 9 lbs per gallon) and bulky. With these weight and space requirements, shipping and storing these containers can be very expensive. Additionally, disposal of these larger containers present environmental issues.

For at least these reasons, recently, there has been a shift in emphasis from providing regular strength detergent formulations to concentrated detergent formulations. Liquid concentrates can reduce manufacturing and shipping costs and require less packaging. Consumers have reacted favorably to the concentrated formulations, and will likely continue their use so long as the products retain their cleaning efficiency.

The surfactant concentrations found in most commercially available concentrates range from about 15% to about 26%. Typically, blends of nonionic and anionic surfactants are used to enable the detergents to remove a variety of stain types. Additional ingredients such as anti-redeposition, builders, enzymes, dye-transferring polymers and foam stabilizers are added to improve detergent performance. Unfortunately, these concentrated detergents are more difficult to process and can result in detergents that undergo phase separation.

There are unbuilt liquid laundry detergents having surfactant concentrations higher than 30%. These detergents typically contain nonionic surfactants. Unbuilt detergents, however, are generally unsuitable for hard water areas and have enjoyed limited success. Further, unbuilt liquid detergents also suffer from instability when the surfactant concentrations are increased.

Shampoo compositions for cleaning hair that also contain conditioning agents are well known as well. Among the preferred types of conditioning agents are nonvolatile, insoluble, fluid conditioning agents such as polydimethylsiloxane and other silicone polymers. Silicone conditioners are particularly valued for their ability to provide a smooth, soft feel to dry hair. Other common insoluble fluid conditioners include organic fluids, e.g., oils, such as hydrocarbons and fatty esters. These conditioners are valued for their ability to replace natural oils lost from the hair due to cleansing surfactants in shampoos, and to add sheen and luster to the hair. In order for these types of shampoos to be effective and to provide a consistent level of performance, without necessitating vigorous shaking of the package in which they are contained, it is necessary to suspend them in the composition with the aid of a suspending agent. Furthermore, since shampoos are likely to remain on shelves or in storage for long periods of time, it is important for the suspending agents to keep the conditioning agents well suspended for long periods of time. The suspending agents which are preferred for suspension of insoluble, dispersed phase fluid conditioning agents are those which form a crystalline network in the shampoo when not exposed to shear, such as when the shampoo is being stored on the shelf, but which allow the composition to be readily flowable when shear is applied, such as when a user tilts a bottle of the shampoo in preparation to dispense a portion of it onto ones hand or hair.

Just as important as suspending the insoluble, dispersed phase fluid conditioning agents, the suspending agent must also allow the conditioning agent to deposit on the hair or scalp during use. If the conditioning agent does not deposit well, large proportions will likely be rinsed away and, therefore, the shampoo will be unable to provide good conditioning efficacy. Alternately, in order to provide good conditioning, relatively high levels of the conditioning agents would need to be incorporated into the shampoo composition. This would add additional cost to the product, reduce lathering, and present additional product stability concerns.

Obtaining good deposition of the conditioning agent is further complicated by the action of detersive, i.e., “cleaning,” surfactants in the shampoo. Detersive surfactants are designed to carry away, or remove, oil, grease, dirt, and particulate matter in general from the hair and scalp. In addition, the detersive surfactants will interfere with deposition of the dispersed phase fluid conditioning agent, and carry away both non-deposited and some deposited conditioning agents during rinsing. This interference makes effective deposition of the conditioning agent even more critical for providing efficacious, cost effective conditioning from a shampoo matrix.

Another important parameter in the formulation of conditioning shampoos is lathering. The consuming public often associates high lathering with effective cleaning, and prefers high lathering shampoos from an aesthetic standpoint. Thus, it has become conventional practice to enhance the lathering performance of these shampoos by increasing the level of or by adding ingredients that promote high lathering.

To circumvent the instability of concentrated liquid detergents, including shampoos, some manufacturers suspend builder and/or other functional materials as solids in liquid surfactant micelles or emulsions. It is difficult to prevent sedimentation of the micelles and emulsions. Other solutions have been proposed. It has been suggested that the addition of counter ions such as ammonium and potassium may increase detergent stability. Nevertheless, no general theory has arisen that explains why some detergent formulations are stable while others separate or become gel like. Thus, there exists a need for economical concentrated and super-concentrated liquid detergents and shampoos that remain stable during storage and transport.

### SUMMARY OF THE INVENTION

The present invention addresses the above-mentioned needs by providing concentrated and super-concentrated compositions for dilution or combination with water to provide a ready-to-use cleanser. The cleanser may be configured or adapted for use as a shampoo, body cleanser, general household and industrial cleaner, window cleaner or laundry detergent and the like.

In accordance with the present invention, there is provided a concentrated composition which, when diluted with water, provides a ready-to-use liquid cleanser or detergent. The concentrated composition includes a suspension matrix and at least one surfactant dispersed in the suspension matrix. The suspension matrix, prior to dilution of the composition, has a concentration of water of less than about 5% by volume. The composition is diluted by the addition of water wherein the resulting concentration of the surfactant is sufficient to function for its intended purpose, i.e., provides the intended cleansing. The type of surfactant employed, the requisite dilution ratio and the requisite concentration of surfactant upon dilution are dependent upon the intended purpose of the cleanser.

The present invention is additionally directed to a method for providing a liquid cleanser, the method including the steps of providing a suspension matrix having a concentration of water of less than about 5% by volume, dispersing at least one surfactant in the suspension matrix, and diluting the suspension matrix with an amount of water wherein the ratio of the suspension matrix to the added water provides a workable volume of cleanser having a sufficient concentration of surfactant to provide the intended cleaning effect. The method of the present invention may further include dispersing at least one active agent in the suspension matrix.

These and other objects, advantages, and features of the invention will become apparent to those persons skilled in the art upon reading the details of the compositions and methods as more fully described below.

### DETAILED DESCRIPTION OF THE INVENTION

Before the present compositions and methods are described, it is to be understood that this invention is not limited to particular compositions and methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a surfactant” includes a plurality of such surfactants and reference to “the detergent” includes reference to one or more detergents and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The present invention is a concentrated composition, e.g., a cleanser, detergent or shampoo, provided for subsequent dilution and combination with water wherein the diluted mixture is in a liquid state and ready to use for its intended purpose, i.e., as a cleanser. In a preferred embodiment, the concentrated composition is provided in a relatively compact and light-weight container or package, e.g., a tube, vial or packet, which can be readily combined with water (such as tap water) by the end user. In its concentrated form, the composition may be a gel or a liquid or any have any other suitable form. Upon dilution with water, the concentrated composition is diluted with water to form an aqueous mixture having a workable volume and a sufficient concentration of surfactant to provide the requisite cleansing effect.

The concentrated composition includes a suspension matrix and at least one surfactant dispersed in the suspension matrix, wherein the suspension matrix has a concentration of water less than about 5% by volume prior to dilution. Depending on the application, the surfactant may be an anionic surfactant, a nonionic surfactant or a cationic surfactant. Suitable surfactants for use with the present invention include but are not limited to sodium lauryl sulfate, cocamidopropyl betaine, lauramidopropylamine oxide, sodium laurel sulfate, and sodium laurel ether sulfate.

As mentioned above, the type of surfactant employed in the concentrated composition, the requisite dilution ratio of the composition and the requisite concentration of surfactant upon dilution are dependent upon the intended purpose of the cleanser. For purposes of illustration only and without limitation, the following are examples of embodiments of the present invention. A concentrated composition of the present invention for use as a shampoo may employ a surfactant such as sodium lauryl sulfate and/or cocamidopropyl betaine requiring a dilution (composition to water) ratio of no less than about 1:13 to provide a surfactant concentration (after dilution) of no less than about 5%. A concentrated composition of the present invention for use as a body cleanser may employ a surfactant such as sodium lauryl ether sulfate requiring a dilution ratio of no less than about 1:13 to provide a surfactant concentration (after dilution) of no less than about 4%. A concentrated composition of the present invention for use as an all-purpose household or industrial detergent may employ a surfactant such as sodium alkyl benzene sulfonate requiring a dilution ratio of from about 1:50 to about 1:256 to provide a surfactant concentration (after dilution) of no less than about 1%. A concentrated composition of the present invention for use as a laundry detergent may employ a surfactant such as triethanolamine dodecyl benzene sulfonate requiring a dilution ratio from about 1:256 to provide a surfactant concentration (after dilution) of no less than about 1%. A concentrated composition of the present invention for use as a window cleaner may employ a surfactant such as sodium lauryl sulphate requiring a dilution ratio of no less than about 1:256 to provide a surfactant concentration (after dilution) of no less than about 0.1%.

The suspension matrix may further include at least one viscosity modifier, at least one fragrance or at least one dye. Suitable viscosity modifier for use with the present invention may include but is not limited to sodium chloride, ammonium chloride and potassium chloride. Whether or not a fragrance or dye is employed may depend on the application of the concentrated liquid composition. For example, if composition is a shampoo for human use, the product is likely to be more marketable if it is appropriately fragrant (e.g., floral, citrus, or herbal fragrances) and/or colored. However, if the shampoo is for veterinary use, no fragrance or a more subtle fragrance may be preferable so as not to be offensive to the animal yet appealing to the animal's groomer. While fragrance and color may not be as vital to the marketability of a household or industrial detergent, they are preferably not offensive to the user.

The concentrated liquid composition of the present invention may further include at least one active agent which is also dispersed within the suspension matrix. A variety of active agents may be employed depending on the intended purpose of the concentrated liquid composition. For example, where the concentrated liquid composition is a shampoo or other cleanser for human or animal use, the active agent may be a topical formulation or medication for treating such conditions as dandruff and fungal infections, or a pesticide for removing parasites. The topical medication used may be an anti-inflammatory, an antifungal, an antibiotic and/or an anti-irritant.

The present invention also provides a method for providing a concentrated liquid cleanser. A preferred embodiment of the subject method includes the steps of providing a suspension matrix as described above, dispersing at least one surfactant as described above in the suspension matrix, and adding an amount of water to the suspension matrix to provide an aqueous mixture having a dilution ration and surfactant concentration as described above. The method may further include the step of dispersing at least one active agent as described above in the suspension matrix.

### EXAMPLE

The following example is put forth so as to provide those of ordinary skill in the art with a complete disclosure and description of how to make and use the present invention, and is not intended to limit the scope of what the inventor regards as the invention. Efforts have been made to ensure accuracy with respect to numbers used (e.g., weights, etc.) but some experimental errors and deviations should be accounted for. Unless indicated otherwise, parts are parts by weight.

An example of a shampoo product according to the present invention includes the following ingredients by weight and percent of the total product:

| Ingredient Type | Ingredient Name | Weight (g) | % of Total |
|---|---|---|---|
| Surfactant | Sodium lauryl ether sulfate | 292.5 | 65.0 |
| Surfactant | Amid | 67.5 | 15.0 |
| Surfactant | Cocamidopropyl betaine | 45.0 | 10.0 |
| pH Modifier | Citric acid | 3.15 | 0.7 |
| Viscosity Modifier | Sodium chloride | 32.3442 | 7.1876 |
| Modifier | DMM hydantoin | 1.35 | 0.3 |
| Modifier | Tetra sodium EDTA | 1.35 | 0.3 |
| Dye | Yellow #5 dry | 0.0279 | 0.0062 |
| Dye | Red #40 dry | 0.0279 | 0.0062 |
| Fragrance | Baby powder fragrance | 6.75 | 1.5 |
| Total | | 450.00 | 100.00 |

Although only a few exemplary embodiments of the present invention have been described in detail above, those skilled in the art will readily appreciate that numerous modifications are to the exemplary embodiments are possible without materially departing from the novel teachings and advantages of this invention. In addition, many modifications may be made to adapt a particular situation, material, composition of matter, process, process step or steps, to the objective, spirit and scope of the present invention. Accordingly, all such modifications are intended to be included within the claims appended hereto.

That which is claimed is:

1. A concentrated composition to be diluted with water, said composition comprising:
a suspension matrix having a concentration of water of less than about 5% by volume prior to dilution; and
at least one surfactant dispersed in said suspension matrix.

2. The concentrated composition of claim 1 wherein said suspension matrix comprises at least one viscosity modifier.

3. The concentrated composition of claim 2 wherein said at least one viscosity modifier is selected from sodium chloride, ammonium chloride and potassium chloride.

4. The concentrated composition of claim 1 wherein said suspension matrix further comprises at least one fragrance.

5. The concentrated composition of claim 1 wherein said suspension matrix further comprises at least one dye.

6. The concentrated composition of claim 1 wherein said at least one surfactant is an anionic surfactant.

7. The concentrated composition of claim 1 wherein said at least one surfactant is a nonionic surfactant.

8. The concentrated composition of claim 1 wherein said at least one surfactant is a cationic surfactant.

9. The concentrated composition of claim 1 wherein said at least one surfactant is selected from sodium lauryl sulfate, cocamidopropyl betaine, lauramidopropylamine oxide, sodium laurel sulfate, and sodium laurel ether sulfate.

10. The concentrated composition of claim 1 further comprising at least one active agent dispersed in said suspension matrix.

11. The concentrated composition of claim 10 wherein said at least one active agent is a topical medication.

12. The concentrated composition of claim 11 wherein said topical medication is selected from an anti-inflammatory, an anti-fungal, an antibiotic and an anti-irritant.

13. The concentrated composition of claim 10 wherein said at least one active agent is a pesticide.

14. The concentrated composition of claim 1 wherein said composition is a detergent.

15. The concentrated composition of claim 1 wherein said composition is a shampoo.

16. The concentrated composition of claim 1, wherein said at least one surfactant is suitable for use as a shampoo and wherein upon dilution of said composition with water, the ratio of said suspension matrix to said water is no less than about 1 to 13 wherein the concentration of said at least one surfactant in said diluted composition is no less than about 5% by volume.

17. The concentrated composition of claim 16, wherein said at least one surfactant is selected from sodium lauryl sulfate and cocamidopropyl betaine.

18. The concentrated composition of claim 1, wherein said at least one surfactant is suitable for use as a body cleanser and wherein upon dilution of said composition with water, the ratio of said suspension matrix to said water is no less than about 1 to 13 wherein the concentration of said at least one surfactant in said diluted composition is no less than about 4% by volume.

19. The concentrated composition of claim 18, wherein said at least one surfactant is sodium lauryl ether sulfate.

20. The concentrated composition of claim 1, wherein said at least one surfactant is suitable for use as an all-purpose detergent and wherein upon dilution of said composition with water, the ratio of said suspension matrix to said water is from about 1 to 50 to about 1 to 256 wherein the concentration of said at least one surfactant in said diluted composition is no less than about 1% by volume.

21. The concentrated composition of claim 18, wherein said at least one surfactant is sodium alkyl benzene sulfonate.

22. The concentrated composition of claim 1, wherein said at least one surfactant is suitable for use as a laundry detergent and wherein upon dilution of said composition with water, the ratio of said suspension matrix to said water is no less than about 1 to 256 wherein the concentration of said at least one surfactant in said diluted composition is no less than about 1% by volume.

23. The concentrated composition of claim 18, wherein said at least one surfactant is triethanolamine dodecyl benzene sulfonate.

24. The concentrated composition of claim 1, wherein said at least one surfactant is suitable for use as a window cleaner and wherein upon dilution of said composition with water, the ratio of said suspension matrix to said water is no less than about 1 to 256 wherein the concentration of said at least one surfactant in said diluted composition is no less than about 0.1% by volume.

25. The concentrated composition of claim 18, wherein said at least one surfactant is sodium lauryl sulphate.

26. A method for providing a liquid cleanser, said method comprising:
providing a suspension matrix having a concentration of water of less than about 5% by volume;
dispersing at least one surfactant in said suspension matrix; and
diluting said suspension matrix with an amount of water.

27. The method of claim 16 further comprising dispersing at least one active agent in said suspension matrix.
